(19) United States
(12) Patent Application Publication
(10) Pub. No.: US 2005/0069961 A1
(43) Pub. Date: Mar. 31, 2005

(54) Isotope-coded affinity tag

(76) Inventors: Oswald Lockoff, Leverkusen (DE); Thomas Lehmann, Wulfrath (DE); Hans-Georg Lerchen, Leverkusen (DE); Dorian Immler, Leverkusen (DE); Hans-Ulrich Siegmund, Leverkusen (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/494,997

(22) PCT Filed: Oct. 28, 2002
(86) PCT No.: PCT/EP02/12006

(30) Foreign Application Priority Data
Nov. 9, 2001 (DE) 10154744.7

(51) Int. Cl. G01N 33/53; C07D 498/02

(52) U.S. Cl. 435/7.5; 530/409; 548/303.1; 536/53

(57) ABSTRACT

The invention relates to novel isotope-coded affinity tags for use in mass-spectrometric analysis of proteins. The invention also relates to the production and use of these novel tags.

The invention relates to novel, isotope-coded affinity tags for the mass-spectrometric analysis of proteins, and to their preparation and use.

Proteomics technology opens up the possibility of identifying novel biological targets and tags by means of analyzing biological systems at the protein level. It is known that only a certain proportion of all the possible proteins encoded in the genome is being expressed at any given time, with, for example, tissue type, state of development, activation of receptors or cellular interactions influencing the pattern and rates of expression. In order to detect differences in the expression of proteins in healthy or diseased tissue, it is possible to make use of a variety of comparative methods for analyzing protein expression patterns ((a) S. P. Gygi et al., Proc. Natl. Acad. Sci. U.S.A., 2000, 97, 9390; (b) D. R. Goodlett et al., Proteome Protein Anal. 2000, 3; (c) S. P. Gygi et al., Curr. Opin. Biotechnol., 2000, 11, 396).

The mass-spectrometric detection of proteins is a powerful method in this connection. When affinity tags which have been isotope-coded differently (ICAT®=isotope coded affinity tags) and tandem mass spectrometry are used, this method can be enlisted for quantitatively analyzing complex protein mixtures ((a) S. P. Gygi et al., Nature Biotechnology, 1999, 17, 994; (b) R. H. Aebersold et al., WO 00/11208). The method is based on each of two or more protein mixtures, which are to be compared and which have been obtained in different cell states, being reacted with an affinity tag of a different isotope coding. After that, the protein mixes are combined, where appropriate fractionated or treated proteolytically and purified by affinity chromatography. After the bound fragments have been eluted, the eluates are analyzed by a combination of liquid chromatography and mass spectrometry (LC-MS). Pairs or groups of peptides which are labeled with affinity tags which only differ in the isotope coding are chemically identical and are eluted virtually simultaneously in the HPLC; however, they differ in the mass spectrometer by the respective molecular weight differences due to the affinity tags having different isotope patterns. Relative protein concentrations can be obtained directly by carrying out measurements of the peak areas. Suitable affinity tags are conjugates composed of affinity ligands which are linked covalently to protein-reactive groups by way of bridge members. In connection with this, different isotopes are incorporated into the bridge members. The method was described using affinity tags in which hydrogen atoms were replaced with deuterium atoms (¹H/²D isotope coding).

However, it has been found that the isotope pair ¹H/²D which is used for coding the described affinity tags cannot be used in an equivalent manner in every case. Pairs of affinity-tagged peptides possessing ¹H/²D isotope coding can be rather different chemically, resulting in a behavior in HPLC which is such that they no longer elute simultaneously. Since matching fragments are now no longer eluted simultaneously, efficient processing of large peptide mixtures is severely limited. Large differences are associated with using affinity tags which are composed either of the ¹H isotopes or the ²D isotopes, with these differences becoming apparent in the differing elution behavior of the given peptide samples.

It has now been found, surprisingly, that the differences between ¹²C-labeled and ¹³C-labeled peptide samples are only marginal and the correspondingly labeled substances exhibit virtually identical elution behavior in HPLC. This labeling method makes parallel manipulation possible, thereby greatly simplifying the analyses. It is now possible, by means of using ¹²C/¹³C isotope-coded affinity tags, to analyze matching fragments from different samples by means of jointly working them up, purifying them and detecting them by mass spectrometry.

The present invention therefore relates to organic compounds, which are suitable for use as affinity tags for the mass-spectrometric analysis of proteins, of the formula (I)
A-L-PRG (I)
in which
A is an affinity ligand residue,
PRG is a protein-reactive group, and
L is a linker which covalently links A and PRG,
which possess at least one carbon atom of the isotope ¹³C.

Preferably, the affinity tags of the formula (I) according to the invention possess two or more carbon atoms of the isotope ¹³C, particularly preferably three, six, nine, twelve, 15, 18, 21 or 24 ¹³C atoms, in particular six, twelve, 18 or 24 ¹³C atoms. In this connection, two or more ¹³C atoms, for example three ¹³C atoms, can be linked to each other by means of “³C—¹³C bonds. It is also possible for several groups of ¹³C atoms which are linked in this way, for example two or more groups in each case possessing three ¹³C atoms, to be present separate from each other in the affinity tag.

The affinity ligand A is used for selectively enriching samples by means of affinity chromatography. The affinity columns are provided with the corresponding reactants which are complementary to the affinity ligands, which reactants enter into covalent or noncovalent bonds with the affinity ligands. An example of a suitable affinity ligand is biotin or a biotin derivative, which enters into strong, noncovalent bonds with the complementary peptides avidin or streptavidin. In this way, it is possible to use affinity chromatography to selectively isolate samples to be investigated from sample mixtures. In the same sense, it is also possible for example, to use carbohydrate residues, which are able to enter into noncovalent interactions with fixed lectins, for example, as affinity ligands. It is furthermore possible to use the interaction of haptens with antibodies, or the interaction of transition metals with corresponding ligands, as complexing agents, or other systems which interact with each other, in the same sense.

Protein-reactive groups, PRG, are used for selectively labeling the proteins at selected functional groups. PRGs have a specific reactivity for terminal functional groups in proteins. Examples of amino acids which, as elements of proteins, are frequently used for selective labeling, are mercaptoaminomonocarboxylic acids, such as cysteine, diaminomonocarboxylic acids, such as lysine or arginine, or monoaminodicarboxylic acids, such as aspartic acid or glutamic acid. Protein-reactive groups can, for example, be thiol-reactive groups such as epoxides, α-haloacyl groups, nitriles, maleimides, sulfonated alkyl or arylthiols. Carboxylate-reactive groups contain amines or alcohols, for example, in the presence of water-extracting agents. Furthermore, protein-reactive groups can also be phosphate-reactive groups, such as metal chelates, and also aldehyde-reactive or ketone-reactive groups, such as amines which, after the formation of a Schiff's base, are, where appropriate, reduced with sodium borohydride or sodium cyanoborohydride. They can also be groups which, after a selective protein derivatization, such as a cyanogen bromide cleavage or an elimination of phosphate groups, etc., react with the reaction products.

Preference is given to compounds according to the invention of the formula (II)
A-B¹-X¹(CH₂)ₙ—[X²—(CH₂)ₘ]ₓX³—(CH₂)ₚ—X
4—B²—PRG (II)
in which
A is an affinity ligand, in particular biotin,
PRG is a protein-reactive group, in particular an epoxide, a maleimido group, a halogen or an acryl residue or another known protein-reactive group, as are described and assembled, for example, by W. H. Scouten in Methods in Enzymology, Volume 135, edited by Klaus Mosbach, Academic Press Inc. 1987, pp. 30ff,
X¹, X², X³ and X⁴ are, independently of each other, and also independently of other X²s in the linker group in the case of X², O, S, NH, NR, CO, CO—O, O—CO, CO—S, S—CO, S—S, SO, SO₂, CO—NR, NR—CO, CS—NR, NR—CS, Si—O or O—Si or an arylene or diarylene group, where X¹, X², X³ or X⁴ may also be absent in some or all cases,
B¹ and B² are, independently of each other, optional fragments which, as bridges, permit or facilitate the linking of A or PRG to the linker L and which, in this connection, may possibly improve the solubility, and are CO—O, O—CO, CO, NR, CO—NR, NR—CO, CS—NR or NR—CS and which contain one or more, preferably between 1 and 10, CH₂ groups, either alone or in combination with other groups, for example (CH₂)_{q}—CONR, (CH₂)_{q}—NR or (CH₂)_{q},
m, n, p, q and x can, independently of each other, be numbers having the respective values of from 0 to 100, where the sum of n+xm+p+q is preferably less than 100 and particularly preferably between 10 and 30, and
R is alkyl, alkenyl, alkynyl, alkoxycarbonyl, aralkoxycarbonyl or aryl.

The corresponding ¹²C-containing compounds of the formulae (I) or (II) are described in WO 00/11208. This latter publication also describes how the compounds of the formulae (I) or (II) can be used for analyzing proteins and protein functions in complex mixtures.

The invention furthermore relates to the use of one or more differently isotope-labeled compounds according to the invention as (a) reagent(s) for the mass-spectrometric analysis of proteins, in particular for identifying one or more proteins or protein functions in one or more protein-containing samples and for determining the relative level of expression of one or more proteins in one or more protein-containing samples.

The present application also describes an improved process for preparing the compounds of the formula (I) or (II), both in the form of the ¹²C-isotope patterns and in the form of those which are labeled with ¹³C.

An important restriction in the use of the compounds mentioned in WO 00/11208 is the difficulty in obtaining them. It is only possible to obtain the affinity tags over a number of steps, and the tags can only be obtained in very low yield in pure form. At the end of the synthesis routes, affinity tags have to be isolated using HPLC purification steps which involve heavy losses. All in all, only unsatisfactory results are achieved when using the described methods to prepare the compounds and, as a result, the isotope-labeled reagents, very particularly, are not readily available, and consequently expensive, thereby militating against any broad use of the method.

It has emerged that the monofunctionalization of α,ω-diaminooxaalkanes is a bottleneck in the synthesis sequences which have been described. Thus, the selective Michael addition of acrylamide derivatives to 1,9-tetradeutero-3,6-dioxa-1,9-nonanediaamine only gives the monosubstitution product in very poor yield (WO 00/11208, page 51; compound 27). In addition, the monofunctionalization of 4,7,10-trioxa-1,13-tridecanediamine with biotin pentafluorophenyl ester, and its subsequent reaction with iodoacetic anhydride to give N-(13-iodoacetamido-4,7-10-trioxatridecanyl)biotinamide (Nature Biotechnology, 1999, 17, 994) only affords the affinity tag reactant in poor yields following an HPLC purification involving heavy losses.

It has been found that the method for preparing the compounds (I) and (II), both in unlabeled form and in ¹³C-labeled form, can be improved if the two amino groups in c,(diaminooxaalkanes, which can serve as linkers L, are initially differentiated by selectively introducing a temporary protecting group (SG) (Scheme 1).

The present invention therefore also relates to a process for preparing an organic compound of the formula (I)
A-L-PRG (I)
in which
A is an affinity ligand residue,
PRG is a protein-reactive group, and
L is a linker which covalently links A and PRG,
by substituting an α,ω-diamine with an affinity ligand residue and a protein-reactive group in arbitrary order,
characterized in that
i) the α,ω-diamine is first of all unilaterally protected with a protecting group SG,
and then either
ii) the unilaterally protected α,ω-diamine is reacted with an affinity ligand to give the intermediate A-L-SG,
iii) the protecting group is removed from the intermediate A-L-SG, and
iv) the intermediate is reacted with a protein-reactive compound to give a compound of the formula (I),
or
ii′) the unilaterally protected a,o-diamine is reacted with a protein-reactive compound to give the intermediate SG-L-PRG,
iii′) the protecting group is removed from the intermediate SG-L-PRG, and
iv′) the intermediate is reacted with an affinity ligand to give a compound of the formula (I).

Suitable protecting groups are alkoxycarbonyl or aralkoxycarbonyl residues which are customary in peptide chemistry, for example methoxycarbonyl (MOC), ethoxycarbonyl (EOC), trichloroethoxycarbonyl, tert-butyloxycarbonyl (BOC), benzyloxycarbonyl or fluorenylmethoxycarbonyl (FMOC), which can be obtained by reacting the corresponding alkyl chloroformates or aralkyl chloroformates in the presence of inorganic or organic bases. The chloroformic acid esters can be reacted with the α,ω-diaminooxaalkanes in equimolar quantities and at a reduced reaction temperature. Suitable temperature ranges are between −78° C. and +20° C., with preferred ranges being between −30° C. and 0° C. The reaction mixture which is obtained in this way can be purified simply, and without any elaborate preparative HPLC purification procedures, by means of partitioning between aqueous and nonpolar organic solvent phases. The polar, unreacted α,ω-diaminooxaalkanes remain in the aqueous phase whereas the L-SG conjugates (monocarbamates) and the SG-L-SG conjugates (dicarbamates), small quantities of which have been formed, are preferentially concentrated in the organic phase. In the case of very long-chain oligoethylene oxides, it can be advantageous to propel the monocarbamate and dicarbamate mixture into the organic phase by means of salting out.

The reaction of the crude mixture of the conjugates L-SG, such as 1 (Scheme 1), with the affinity ligands A can in turn be effected using known methods. Examples of activated affinity ligands are biotin pentafluorophenyl esters or mixed anhydrides composed of biotin and alkyl chloroformates. In this step, too, the reaction mixture can be worked up simply by partitioning between aqueous and organic phases. When this is done, conjugates A-L-SG, such as 2, are obtained in high yields. The selective elimination of the temporary protecting groups SG affords conjugates composed of affinity ligand and ligand, A-L. These reactions can in turn be carried out using known methods.

Taken overall, using protecting groups consequently makes it possible to obtain the conjugates A-L much more easily, and with better yields, than when using the previously described methods.

The protein-reactive groups PRG are introduced into the conjugates A-L using known methods. Reacting A-L with iodoacetic anhydride results in conjugates A-L-PRG which possess an iodoacetamide group as the protein-reactive group (for example 4) with this group selectively reacting with sulfhydryl groups in cysteine side chains of peptides or proteins. A-L-PRG conjugates in which the PRGs are maleimide residues (for example 5 or 6) also react with sulfhydryl groups.

It has furthermore been found that the above-described process for preparing the A-L-PRG conjugates can also advantageously be used when individual ¹²C atoms in the linker units L are replaced with ¹³C atoms (Scheme 2). Relevant chemical steps are directly applicable to the ¹³C building blocks. In this connection, it is particularly advantageous that the ¹³C-labeled intermediates, some of which are very expensive, can be prepared with good yields and once again without any elaborate HPLC purifications.

With their ¹³C isotope labels, the described compounds of the formula (I) are outstandingly suitable, when taken together with their unlabeled analogs, for analyzing complex protein mixtures. In this connection, the ¹²C/¹³C affinity tag pairs differ advantageously from the corresponding ¹H/²D affinity tag pairs which are already described in detail (WO 00/11208 and Nature Biotechnology, 1999, 17, 994). In this connection, it is particularly advantageous that peptide samples which have been labeled either with ¹²C affinity tags or with ¹³C affinity tags are eluted virtually simultaneously in HPLC performed on reverse phase (RP) materials. Consequently, matching samples can also be measured simultaneously in coupled mass spectrometry. On the other hand, comparison samples which have been labeled with ¹H affinity tags or with ²D affinity tags differ significantly in their migratory behavior in HPLC. Peptides which are linked to deuterated affinity tags are eluted markedly sooner in RP-HPLC than are the corresponding ¹H affinity tag-peptide conjugates. These time differences in the ¹H/²D elution behavior, which can be in the two-digit second range, do not permit any simultaneous analysis of matching samples of the same peptide frequency in the mass spectrometer. The advantages of using ¹²C/¹³C affinity tag pairs as compared with the corresponding ¹H/²D affinity tag pairs are made clear by the experiments which have been carried out.

### EXAMPLES

The following synthesis protocols relate to Schemes 1 and 2. In the examples, the ¹³C atoms are marked by *

### Example 1

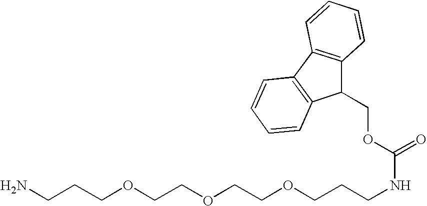

A solution of 9-fluorenylmethyl chloroformate (23.5 g; 90.8 mmol) in tetrahydrofuran (500 ml) was slowly added dropwise, at −10° C., to a solution of 4,7,10-trioxa-1,13-tridecanediamine (20.0 g; 90.8 mmol) in 2-propanol (2000 ml). After 2 h, the mixture was concentrated under reduced pressure. The residue was taken up in dichloromethane (1000 ml) and washed twice with a saturated solution of sodium chloride (250 ml on each occasion). The organic phase was dried and concentrated. The residue is to a large extent homogeneous and free from the starting compound 4,7,10-trioxa-1,13-tridecanediamine. Yield of crude product 36.5 g (91%), syrup. Rf. 0.27 (dichloromethane/methanol=5:1); MS (ESI): m/z 443.4 (M+H)⁺.

### Example 2

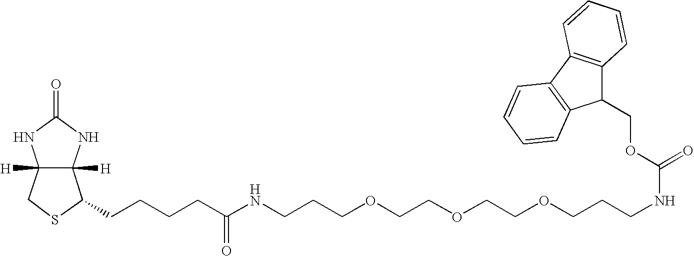

Ethyl chloroformate (590 mg; 5.4 mmol) and N-ethyldiisopropylamine (1.40 g; 10.8 mmol) were added dropwise, at 0° C., to a solution of biotin (1.32 g; 5.4 mmol) in N,N-dimethylformamide (60 ml). After 2 h at 20° C., the mixture was added dropwise to the solution of compound 1 (1.20 g; 2.7 mmol) and the whole was stirred for 1 h. The mixture was concentrated and the residue was taken in dichloromethane; this solution was then washed with 1N hydrochloric acid and a saturated solution of sodium chloride, after which it was dried and concentrated. The residue was purified chromatographically through silica gel (eluent, dichloromethane/methanol, gradient 50:1->10:1). Yield 1.4 g (77%), syrup. Rf 0.51 (dichloromethane/methanol=5:1); MS (ESI): m/z 669.4 (M+H)⁺.

### Example 3

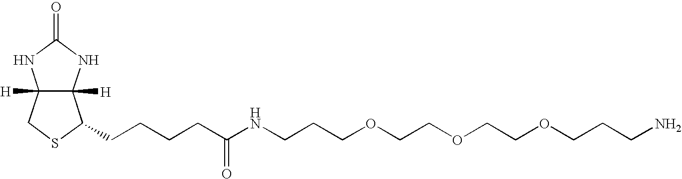

Piperidine (2.7 g; 31.9 mmol) was added to a solution of compound 2 (1.0 g; 1.50 mmol) in methanol (80 ml). After 16 h at 20° C., the mixture was concentrated. The residue was taken up in water and this solution was washed 5 times with dichloromethane. The aqueous phase was lyophilized. Yield 0.53 g (79%), amorphous solid. Rf 0.13 (acetonitrile/water/acetic acid=10:3:0.1); MS (ESI): m/z 447.3 (M+H)⁺.

### Example 4

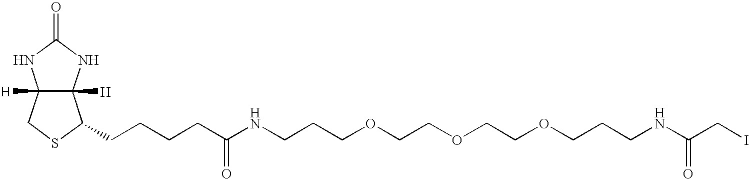

Iodoacetic anhydride (87.2 mg; 0.25 mmol) was added to a solution of compound 3 (100 mg; 0.22 mmol) in dimethylformamide (5 ml). After 1 h, the mixture was concentrated. The residue was taken up in methanol (5 ml) and this solution was stirred together with a weakly basic anion exchanger in order to remove any remaining iodoacetic acid. The resin was filtered off with suction and the filtrate was concentrated. The residue was purified by column chromatography (eluent, dichloromethane/methanol=30:1). Yield 64 mg (46%), syrup. Rf 0.40 (dichloromethane/methanol=5:1); MS (ESI): m/z 615.3 (M+H)⁺.

### Example 5

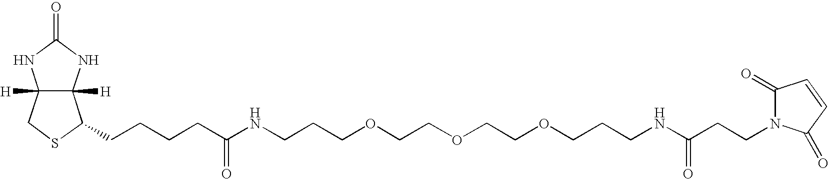

Maleimidopropionic acid (13 mg; 0.078 mmol) was dissolved in DMF (5 ml), after which compound 3 (35 mg; 0.078 mmol), and also 1-hydroxy-1H-benzotriazole (16 mg; 0.118 mmol), N-(3-dimethylaminopropyl)-N′-ethylcarbodiimide hydrochloride (18 mg; 0.094 mmol) and Hünig base (30 mg), were added. The mixture was stirred overnight at room temperature and concentrated and the residue was purified by flash chromatography under silica gel (elution mixture acetonitrile/water=10/1). The appropriate fractions were combined and the solvent was evaporated off in vacuo. The residue was precipitated with diethyl ether from dichloromethane/methanol. Yield, 8 mg (17%), amorphous solid. Rf 0.12 (acetonitrile/water 10:1); MS (ESI): m/z 598 (M+H)⁺.

### Example 6

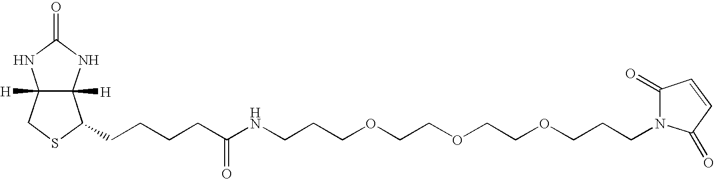

Compound 3 (22.3 mg; 0.05 mmol) was initially introduced in DMF (10 ml) and maleic anhydride (4.9 mg; 0.05 mmol) was then added. The mixture was stirred overnight at RT. 1-Hydroxy-1H-benzotriazole (0.075 mmol), N-(3-dimethylaminopropyl)-N′-ethylcarbodiimide hydrochloride (0.06 mmol) and Hünig base (50 mg) were then added. The mixture was stirred at RT for a further 2 days. It was concentrated and the residue was purified by flash chromatography (eluent, acetonitrile/water=10:1).

### Example 7

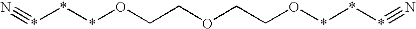

Diethylene glycol (3.18 g; 30 mmol) was added dropwise to a solution of aqueous potassium hydroxide (40%; 120 mg) and 1,4-dioxane (3.75 ml). 1,2,3-¹³C-acrylonitrile (3.36 g; 60 mmol) was added dropwise to the solution while cooling it with ice. After the solution had been warmed to room temperature, it was stirred for a further 16 h. The solution was diluted with dichloromethane (30 ml) and washed twice with a saturated solution of sodium chloride. The organic phase was dried and concentrated. The residue was purified by column chromatography (eluent: dichloromethane/methanol=50:1). Yield 6.21 g (95%), syrup. Rf 0.71 (dichloromethane/methanol=10:1); MS (ESI): m/z 241.1 (M+Na)⁺, 219.1 (M+H)⁺.

### Example 8

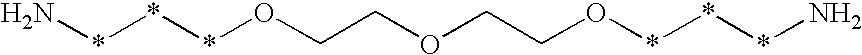

Raney nickel (2.5 g) was added to a solution of compound 7 (5.0 g; 22.9 mmol) in methanol (115 ml) and a concentrated aqueous solution of ammonia (68 ml) and the mixture was hydrogenated with hydrogen for 5 h at 100° C. and 100 bar. After the mixture cooled down to room temperature, the catalyst was filtered off with suction. The filtrate was concentrated. The residue was taken up three times in ethanol, with this solution being concentrated. Yield 3.84 g (74%), syrup. Rf=0.27 (dichloromethane/methanol/ammonia=4:3:1); MS (ESI): m/z 227.3 (M+H)⁺; ¹³C-NMR (100.6 MHz, CDCl₃): δ=69.48, 69.10 (¹³CH₂—O), 39.13, 38.77 (¹³CH₂—NH₂), 32.74, 32.38, 32.36, 32.01 (¹³CH₂—¹³CH₂—¹³CH₂).

### Example 9

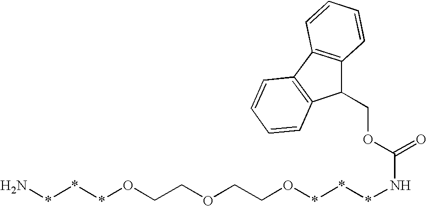

A solution of 9-fluorenyl chloroformate (1.14 g; 4.42 mmol) in tetrahydrofuran (20 ml) was added, at −10° C., to a solution of compound 8 (1.0 g; 4.42 mmol) in 2-propanol (50 ml). The mixture was slowly warmed to room temperature and stirred for 16 h. It was then concentrated. The residue was taken up in dichloromethane (50 ml) and this solution was washed twice with a saturated aqueous solution of sodium chloride, after which it was dried and concentrated. The residue was to a large extent homogeneous and was used in the following step without any further purification. Yield 1.76 g (89%), syrup. Rf=0.27 (dichloromethane/methanol=5:1); MS (ESI): m/z 449.4 (M+H)⁺.

### Example 10

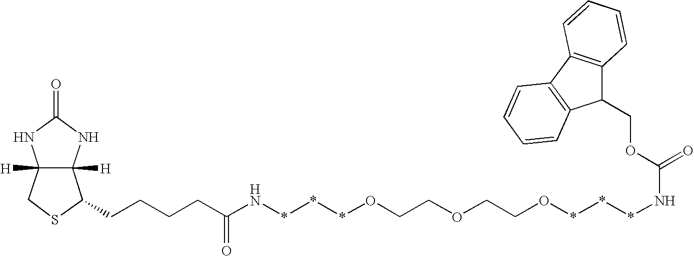

Ethyl chloroformate (959 mg; 8.8 mmol) and N-ethyldiisopropylamine (2.29 g; 17.7 mmol) were added dropwise, at 0° C., to a solution of biotin (2.16 g; 8.8 mmol) in N,N-dimethylformamide (100 ml). After 2 h at 20° C., the mixture was added dropwise to the solution of the crude product 9 (1.76 g; 3.9 mmol) and the whole was stirred for 1 h. The mixture was concentrated and the residue was taken up in dichloromethane and this solution was washed with 1N hydrochloric acid and a saturated solution of sodium chloride, after which it was dried and concentrated. The residue was purified chromatographically through silica gel (eluent, dichloromethane/methanol, gradient 50:1->10:1). Yield 1.6 g (61%), syrup. Rf=0.51 (dichloromethane/methanol=5:1); MS (ESI): m/z 675.3 (M+H)⁺; ¹³C-NMR (100.6 MHz, CDCl₃): δ=69.95, 69.67, 69.49, 69.11 (¹³CH₂—O), 39.08, 38.71, 37.74, 37.38 (¹³CH₂—NH₂), 29.69, 29.32, 29.21, 28.95, 28.83, 28.46 (¹³CH₂—¹³CH₂—¹³CH₂).

### Example 11

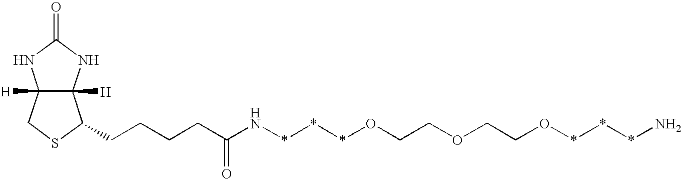

Compound 10 (1.18 g; 1.74 mmol) was dissolved in methanol (20 ml) and piperidine (1 ml) was added. After 2 h at room temperature, the mixture was concentrated. The residue was taken up in water (20 ml) and this solution was washed 5 times with dichloromethane. The aqueous phase was lyophilized. Yield 905 mg (88%), amorphous solid. MS (ESD): m/z 453.4 (M+H)⁺.

### Example 12

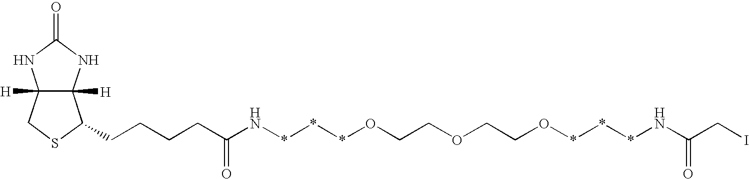

Iodoacetic anhydride (86 mg; 0.24 mmol) was added to a solution of compound 11 (100 mg; 0.22 mmol) in dimethylformamide (5 ml). After 2 h, the mixture was concentrated and the residue was purified by means of preparative HPLC. Yield 101 mg (73%), syrup. Rf=0.37 (dichloromethane/methanol=5:1); MS (ESI): m/z 621.2 (M+H)⁺; 643.2 (M+Na)+; ¹³C-NMR (100.6 MHz, CDCl₃): δ=69.94, 69.83, 69.56, 69.45 (¹³CH₂—O), 38.62, 38.26, 37.72, 37.36 (¹³CH₂—NH₂), 29.22, 28.85, 28.71, 28.48, 28.34, 28.33, 27.96 (¹³CH₂—¹³CH₂—³CH₂).

### Example 13

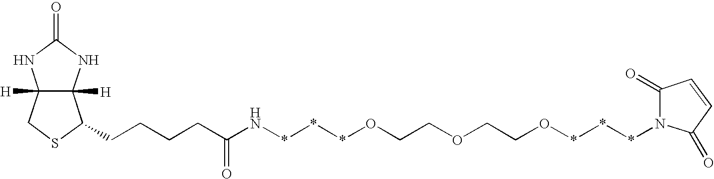

Compound 11 (22.6 mg; 0.05 mmol) was initially introduced in DMF (10 ml) and maleic anhydride (4.9 mg; 0.05 mmol) was added. The mixture was stirred overnight at RT. 1-Hydroxy-1H-benzotriazole (0.075 mmol), N-(3-dimethylaminopropyl)-N′-ethylcarbodiimide hydrochloride (0.06 mmol) and Hünig base (50 mg) were then added. The mixture was stirred at RT for a further 2 days. It was concentrated and the residue was purified by flash chromatography (eluent, acetonitrile/water=10:1).

Biological Investigations

Coupling the Affinity Tags to SDS-7

A mixture of seven proteins, which is also used as a size standard in gel electrophoresis (SDS-7 markers, Sigma-Aldrich GmbH, Taufkirchen), was used as the sample.

24 μg of the protein mixture were dissolved in 5 μl of buffer 1 (50 mM tris-HCl, pH 8.3; 5 mM EDTA; 0.5% (w/v) SDS) and this solution was diluted with 45 μl of buffer 2 (50 mM tris-HCl, pH 8.3; 5 mM EDTA). The proteins were denatured by heating the solution at 100° C. for 3 minutes. In order to reduce the cysteines which were present, 0.8 μl of reducing solution (10% (w/v) tributylphosphine in isopropanol) was added and the -mixture was incubated at 37° C. for 30 minutes. In order to react the free cysteines with affinity tags, 10 μl of derivatizing solution (30 μg of the affinity tags according to the invention or of their ¹²C analogs/μl in DMSO) were then added and the mixture was incubated at 37° C. for 90 minutes.

Two identical SDS-7 samples were prepared as described above, as test samples, and derivatized with the unlabeled affinity tag from Example 4 and the ¹³C-labeled affinity tag from Example 12. The commercially available D₀/D₈-ICAT®s (Applied Biosystems, Foster City) were also used for comparison.

After the derivatization, in each case 20 μl of the two samples to be compared, which samples had previously been treated separately, were mixed and 1 μl of trypsin solution (1 mg of trypsin (Promega GmbH, Mannheim)/ml in buffer 2) was added. The proteins were cleaved overnight (approx. 17 hours) at 37° C.

Affinity Purification of Derivatized Peptides

Derivatized peptides were affinity-purified on freshly prepared affinity columns (Monomeric Avidin, Perbio Science Deutschland GmbH, Bonn) which had a column volume of 200 μl and which were prepared by means of the following washing steps: a) two column volumes of 2×PBS; b) four column volumes of 30% (v/v) acetonitrile/0.4% (v/v) trifluoroacetic acid; c) seven column volumes of 2×PBS; d) four column volumes of 2 mM biotin in 2×PBS; e) six column volumes of 100 mM glycine, pH, 2.8 and f) six column volumes of 2×PBS.

Prior to loading, the sample (30 μl) was diluted with 30 μl of 2×PBS and this diluted solution was then loaded onto the column. The following washing steps were then carried out in order to remove the unbiotinylated peptides: a) six column volumes of 2×PBS; b) six column volumes of PBS; c) six column volumes of 50 mM ammonium hydrogen carbonate/20% (v/v) methanol and d) one column volume of 0.3% (v/v) formic acid. The sample was eluted by the following steps: a) three column volumes of 0.3% (v/v) formic acid and b) three column volumes of 30% (v/v) acetonitrile/0.4% (v/v) trifluoroacetic acid. The eluate was evaporated to dryness and not redissolved until shortly before the mass-spectrometric analysis.

Mass-Spectrometric Analysis

An ion trap mass spectrometer (ThermoFinnigan, San Jose), which was directly connected to a high pressure liquid chromatography appliance (LC-MS), was used for analyzing the peptides. A reversed-phase column (C₁₈ phase) was employed as the separating column. The peptides were dissolved in eluent A (0.025% (v/v) trifluoroacetic acid) and injected. They were eluted with a gradient of eluent B (0.025% (v/v) trifluoroacetic acid, 84% (v/v) acetonitrile). The eluting peptides were automatically recognized by the acquisition software in the unit and fragmented for identification. In this way, it was possible to determine the identity of the peptides unambiguously.

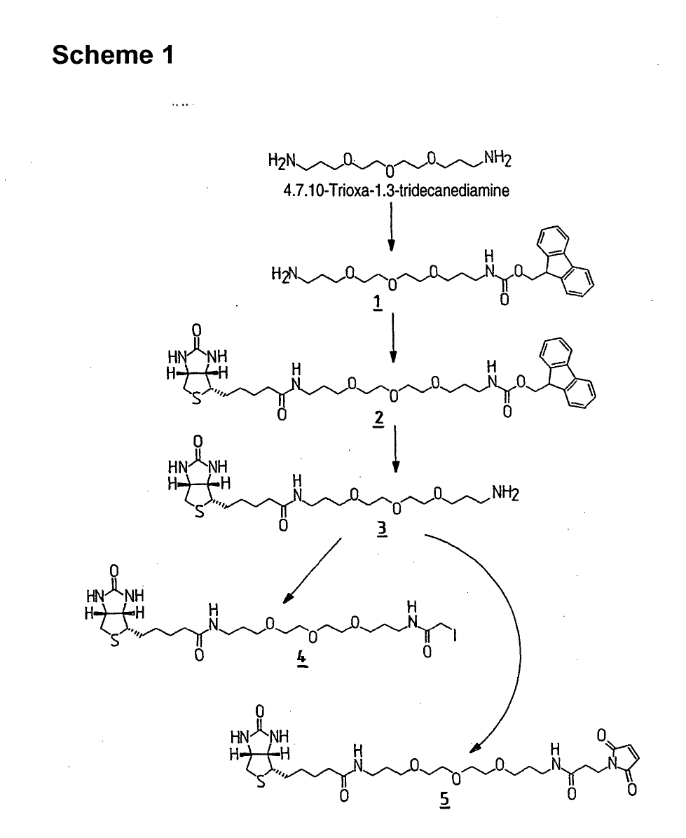

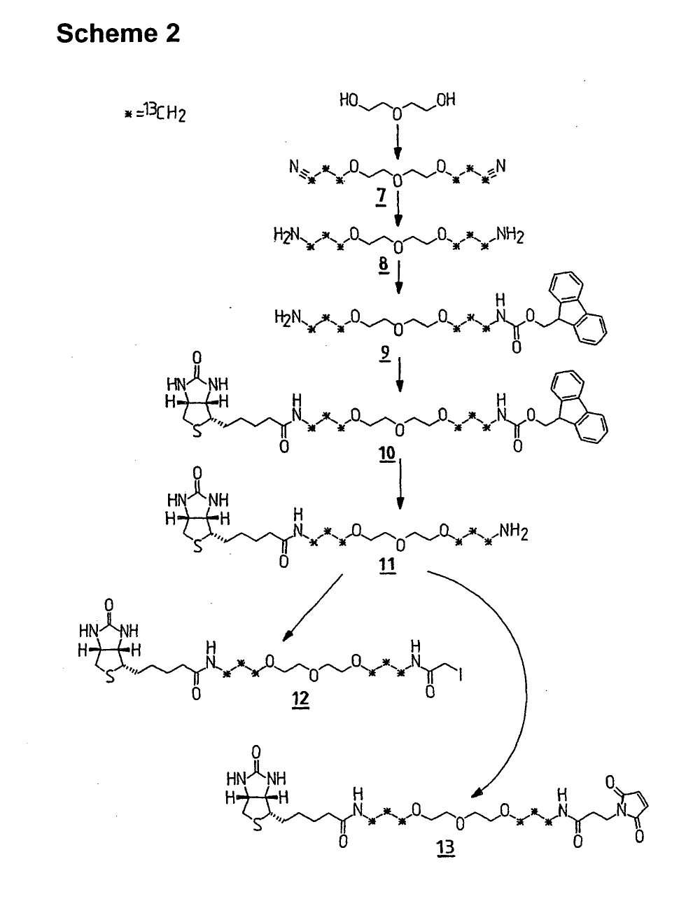

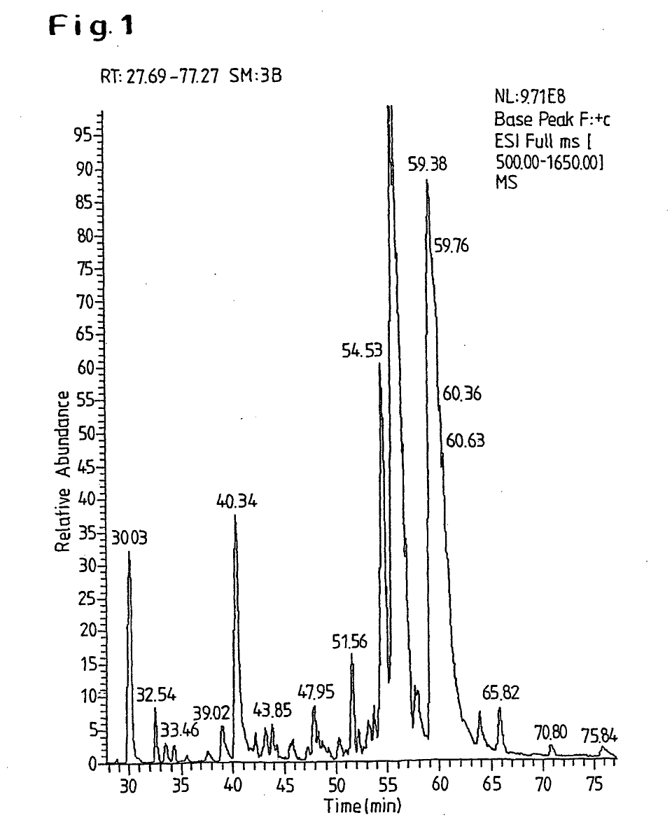

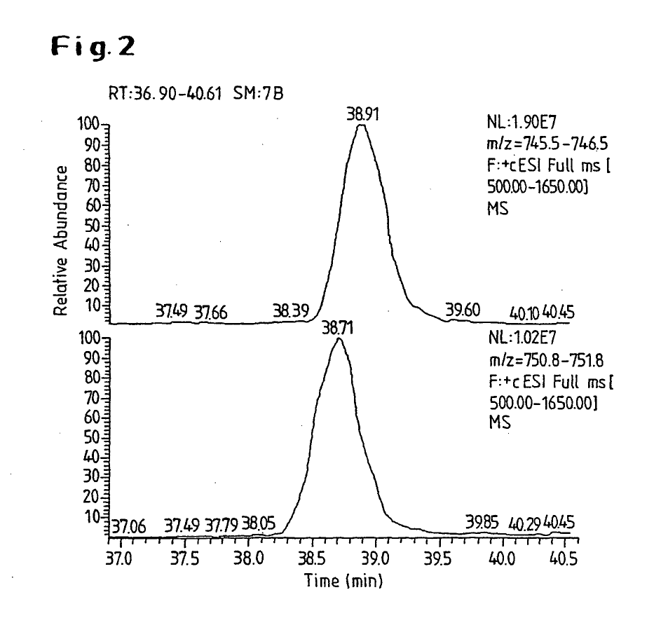

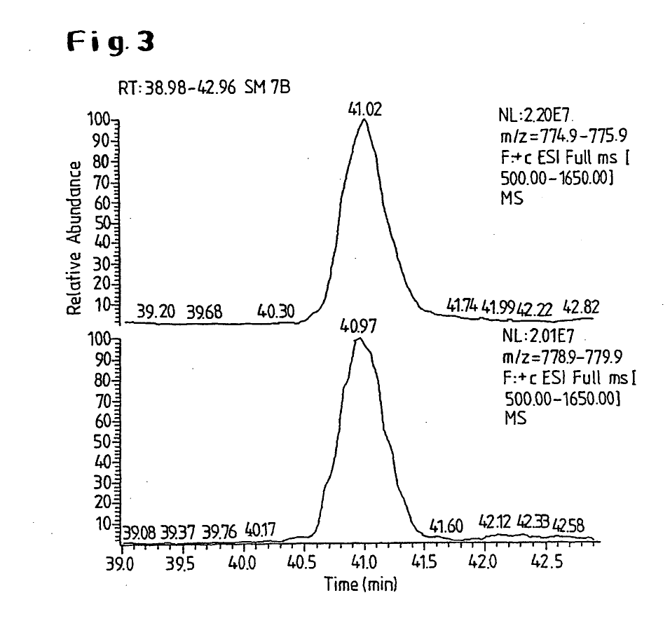

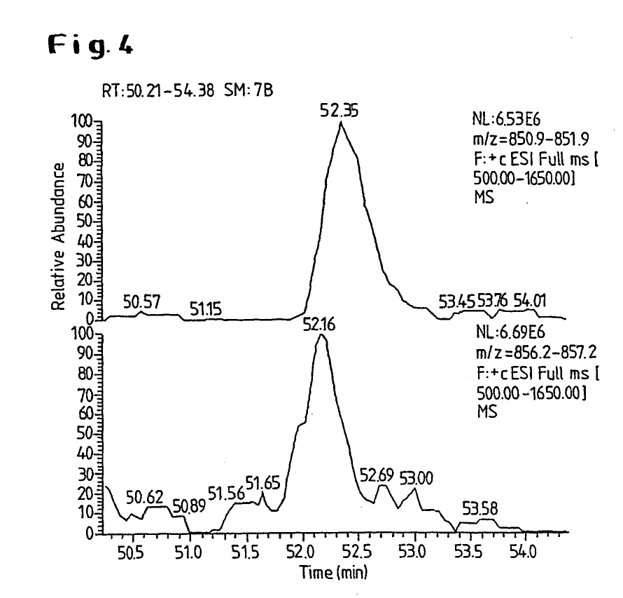

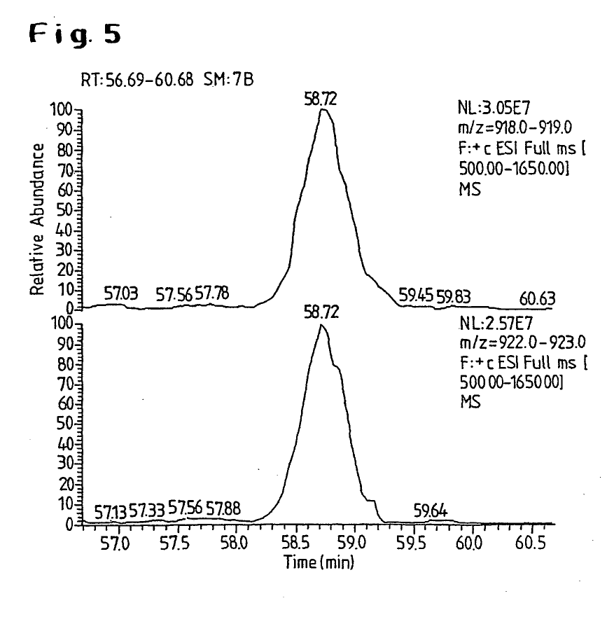

FIG. 1 shows a chromatogram as is customary for peptide mixtures. A large number of peptides are being eluted. FIGS. 2 to 5 in each case depict the ion traces for two peptide pairs. The intensity of the signals is plotted in a mass range which corresponds to the three-fold positively charged peptide ions. It can be clearly seen in FIGS. 2 and 4 that the light and heavy variant of a peptide-ICAT® conjugate do not coelute when the derivatization is carried out using the unlabeled (D₀) and the 8-fold deuterium-labeled (D₈) ICAT®. The differences in migration time amount to from 0.2 to 0.5 minutes depending on the size of the peptide and the number of cysteines in the sequence, with the deuterium-containing form in each case eluting sooner. FIGS. 3 and 5 show the same peptide pairs which have been derivatized with the unlabeled (¹³C₀) affinity tag from Example 4 and the 6-fold ¹³C-labeled (¹³C₆) affinity tag from Example 12. It can be seen that in these instances the two peptides coelute perfectly. In every case, the sequences of the peptides were confirmed by the fragmentation patterns.

### FIG. 1: Chromatogram of an affinity tag-derivatized sample.

FIG. 2: Ion traces of the light and heavy variants of the bovine serum albumin peptide TCVADESHAGCEK (triply charged peptide ions) when using D₀/D₈-ICAT®s.

FIG. 3: Ion traces of the light and heavy variants of the bovine serum albumin peptide TCVADESHAGCEK (triply charged peptide ions) when using the ¹³C₀/¹³C₆ affinity tags from Examples 4 and 12.

FIG. 4: Ion traces of the light and heavy variants of the bovine lactalbumin peptide ALCSEKLDQWLCEK (triply charged peptide ions) when using D₀/D₈-ICAT®s.

FIG. 5: Ion traces of the light and heavy variants of the bovine lactalbumin peptide ALCSEKLDQWLCEK (triply charged peptide ions) when using the ¹³C₀/¹³C₆ affinity tags from Examples 4 and 12.

1. An organic compound of the formula (I),
A-L-PRG (I)
in which
A is an affinity ligand residue,
PRG is a protein-reactive group, and
L is a linker which covalently links A and PRG,
characterized in that it possesses at least one carbon atom of the isotope ¹³C.

2. A compound as claimed in claim 1, characterized in that the protein-reactive group PRG exhibits a selective reactivity for an amino acid group, a phosphate group, an aldehyde group or a keto group of the protein or for a product of a reaction of the protein.

3. A compound as claimed in claim 1 or 2, characterized in that the protein-reactive group-PRG is an epoxide, a maleimido group, a halogen or an acryl residue.

4. A compound as claimed in one of the preceding claims, characterized in that the affinity ligand residue A is the residue of biotin, of a biotin derivative, of a carbohydrate, of a hapten or of a sequestering agent, in particular of biotin or of a biotin derivative.

5. A compound as claimed in one of the preceding claims, characterized in that it satisfies the formula (II)
A-B¹-X¹(CH₂)ₙ—[X²—(CH₂)ₘ]ₓ—X³—(CH₂)ₚ—X⁴—B²—PRG (II)
in which
A is an affinity ligand, in particular biotin,
PRG is a protein-reactive group, in particular an epoxide, a maleimido group, a halogen or an acryl residue,
X¹, X², X³, X⁴ are, independently of each other, and also independently of other X²s in the linker group in the case of X², O, S, NH, NR, CO, CO—O, O—CO, CO—S, S—CO, S—S, SO, SO₂, CO—NR, NR—CO, CS—NR, NR—CS, Si—O or O—Si or an arylene or diarylene group, where X¹, X², X³ or X⁴ may also be absent in some or all cases,
B¹ and B² are, independently of each other, optional fragments which, as bridges, permit or facilitate the linking of A or PRG to the linker L and which, in this connection, may possibly improve the solubility, and are CO—O, O—CO, CO, NR, CO—NR, NR—CO, CS—NR or NR—CS and which contain one or more, preferably between 1 and 10, CH₂ groups, either alone or in combination with other groups, for example (CH₂)_{q}—CONR, (CH₂)_{q}—NR or (CH₂)_{q},
m, n, p, q and x can, independently of each other, be numbers having the respective values of from 0 to 100, where the sum of n+xm+p+q is preferably less than 100 and particularly preferably between 10 and 30, and
R is alkyl, alkenyl, alkynyl, alkoxycarbonyl, aralkoxycarbonyl or aryl.

6. A process for preparing an organic compound of the formula (I)
A-L-PRG (I)
in which
A is an affinity ligand residue,
PRG is a protein-reactive group, and
L is a linker which covalently links A and PRG,
by substituting an α,ω-diamine with an affinity ligand residue and a protein-reactive group in arbitrary order,
characterized in that
i) the α,ω-diamine is first of all unilaterally protected with a protecting group SG,
and then either
ii) the unilaterally protected α,ω-diamine is reacted with an affinity ligand to give the intermediate A-L-SG,
iii) the protecting group is removed from the intermediate A-L-SG, and
iv) the intermediate is reacted with a protein-reactive compound to give a compound of the formula (I),
or
ii′) the unilaterally protected α,ω-diamine is reacted with a protein-reactive compound to give the intermediate SG-L-PRG,
iii′) the protecting group is removed from the intermediate SG-L-PRG, and
iv′) the intermediate is reacted with an affinity ligand to give a compound of the formula (I).

7. The process as claimed in claim 6, characterized in that an α,ω-diaminooxaalkane is used as α,ω-diamine.

8. The process as claimed in claim 6 or 7, characterized in that the α,ω-diamine is protected with an alkoxycarbonyl residue or aralkoxycarbonyl residue as the protecting group SG, preferably with methoxycarbonyl (MOC), ethoxycarbonyl (EOC), trichloroethoxycarbonyl, tert-butyloxycarbonyl (BOC), benzyloxycarbonyl or fluorenylmethoxycarbonyl (FMOC).

9. The process as claimed in one of claims 6 to 8, characterized in that use is made of an α,ω-diamine which possesses at least one carbon atom of the isotope ¹³C.

10. The use of one or more differently isotope-labeled compounds as claimed in one of claims 1 to 5, or obtained by the process as claimed in one of claims 6 to 9, as (a) reagent(s) for the mass-spectrometric analysis of proteins.

11. The use as claimed in the preceding claim for identifying one or more proteins or protein functions in one or more protein-containing samples.

12. The use as claimed in claim 10 for determining the relative level of expression of one or more proteins in one or more protein-containing samples.
